(19) United States
(12) Patent Application Publication
(10) Pub. No.: US 2024/0376343 A1
(43) Pub. Date: Nov. 14, 2024

(54) POLYUREA COMPOSITIONS FROM POLYASPARTIC ESTERS AND 2-SUBSTITUTED BUTANEDIOIC ACID ESTERS

(71) Applicant: EVONIK OPERATIONS GMBH, Essen (DE)

(72) Inventors: Gauri Sankar Lal, Whitehall, PA (US); Shafiq Fazel, Allentown, PA (US); Stephen Michael Boyce, Bath, PA (US)

(73) Assignee: EVONIK OPERATIONS GMBH, Essen (DE)

(21) Appl. No.: 18/683,164

(22) PCT Filed: Jul. 15, 2022
(86) PCT No.: PCT/EP2022/069848

§ 371 Date: Feb. 12, 2024

Related U.S. Application Data
(60) Provisional application No. 63/234,092, filed on Aug. 17, 2021.

(51) Int. Cl. C09D 175/02; C07C 253/18; C07C 253/30; C08G 18/32; C08G 18/73; C08K 5/315; C09D 7/63

(52) U.S. Cl. CPC C09D 175/02; C07C 253/18; C07C 253/30; C08G 18/3234; C08G 18/73; C08K 5/315; C09D 7/63

(57) ABSTRACT

The present invention relates to a polyurea coating composition comprising (A) a polyisocyanate: (B) a polyaspartic ester; and (C) a 2-substituted butanedioic acid ester prepared by reacting the fumaric ester in the polyaspartic acid ester solution with a cyanoacetate, a malononitrile or a 1,3-diketone in the presence of a base.

### BACKGROUND OF THE INVENTION

Polyurea coating compositions having a polyisocyanate binder component and a polyaspartic ester curing agent for reaction with polyisocyanate are known in the art. They have been used for the preparation of coatings that are resistant to weathering, abrasion and solvents. In addition, they may be made rigid and elastic.

Polyaspartic esters which contain secondary polyamines in combination with lacquer polyisocyanates are described in EP-A-0,403,921 for the preparation of coatings without solvent or with a minimal amount of solvent. In EPA-A-0.531,249 coating compositions of polyaspartic esters are used in combination with hydroxyl-containing resins, polyaldimines or ketimines as the iso-cyanate reactive component.

Polyaspartic esters are generally prepared by the reaction of a polyamine with a dialkyl ester of maleic or fumaric acid. If the preparation starts with the maleic acid ester there is a rapid isomerization to the fumaric acid ester which ‘then reacts with the polyamine to generate the polyaspartic ester. The reaction of polyamines with the generated fumaric ester is much slower than with maleic ester. Consequently, excess starting materials are always present after the production of polyaspartic esters. In U.S. Pat. No. 5,236,741 and U.S. Pat. No. 5,623,045 the starting materials were removed by distillation. This is an expensive and laborious process.

In our laboratory we have found that the polyaspartic ester prepared from diethyl maleate/fumarate with bis (4-aminocyclohexyl) methane in a 1:1 molar ratio of reactants takes several weeks to achieve near quantitative conversion of the maleate/fumarate starting material. A similar process used to prepare the polyaspartic ester using (3-methyl-4-aminocyclohexyl)methane took at least three months to achieve near quantitative conversion. As a result, the use of these products in commerce is severely hampered as customers have to wait a very long time between manufacture and receipt of products. In addition, it has been reported that diethyl fumarate is an irritant and hence can affect the health of workers using these materials.

U.S. Pat. No. 6,737,500 describes a method for removing the excess maleic/fumaric ester from the polyaspartic ester production process by carrying out the reaction in two-steps. After an initial reaction of a cyclic amine with an ester of fumaric or maleic acid, an acyclic amine was added to react with the excess fumaric or maleic ester. A similar process was used in U.S. Pat. No. 6,590,066 B1 to remove the excess fumaric/maleic ester using an acyclic amine after an initial reaction of the maleic/fumaric acid ester with a polypropylene oxide amine. These patents describe the production of polyaspartic ester mixtures. The polyaspartic ester derived from cyclic amines and polypropylene oxide amines react much slower with polyisocyanates than those derived from acyclic amines. Hence the working time or pot life of these mixtures are much reduced in coatings applications. This can cause pre-mature gelation of the polyurea coatings and resulting difficulty in application of such mixtures to the substrate to be coated.

There is a need in this art for polyaspartic esters without fumaric or maleic acid esters contaminants which can be produced in a timely and efficient manner and which can be used for production of polyurea coatings without affecting the cured properties of these coatings.

### BRIEF SUMMARY OF THE INVENTION

This invention relates to a polyurea coating composition comprising
(A) a polyisocyanate;
(B) a polyaspartic acid ester represented by the structure below:

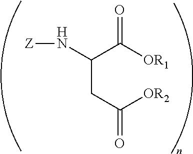

Z=a cycloalkyl or alkyl group, R₁, R₂=alkyl groups containing 1-10 carbon atoms, and n=2-4; and
(C) a 2-substituted butanedioic acid ester prepared by reacting the fumaric ester in the polyaspartic acid ester solution with a cyanoacetate, a malononitrile or a 1,3-diketone in the presence of a base.

This invention also relates to a process for the preparation of a polyurea coating by using a mixture of the following components:
(A) a polyisocyanate;
(B) a polyaspartic acid ester represented by the structure below:

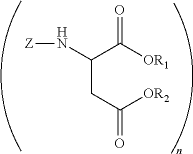

Z=a cycloalkyl or alkyl group, R₁, R₂=alkyl groups containing 1-10 carbon atoms, and n=2-4; and
(C) a 2-substituted butanedioic acid ester prepared by reacting the fumaric ester in the polyaspartic acid ester solution with a cyanoacetate, a malononitrile or a 1,3-diketone in the presence of a base.

This invention also relates to a process for preparing a polyaspartic/2-substituted butanedioic acid ester mixture in-situ by initially reacting an ester of fumaric acid or maleic acid with a polyamine and then reacting the residual ester of fumaric acid or maleic ester to completion with a cyanoacetate, or a malononitrile or a 1,3-diketone in the presence of a base. The method makes it possible to prepare polyurea coatings without maleic acid or fumaric acid esters thereby providing a safer and more environmentally friendly product.

### DETAILED DESCRIPTION OF INVENTION

This invention relates to a polyurea coating composition comprising
(A) a polyisocyanate
(B) a polyaspartic acid ester represented by the structure below:

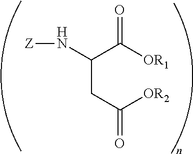

Z=a cycloalkyl or alkyl group, R₁, R₂=alkyl groups containing 1-10 carbon atoms, and n=2-4; and
(C) a 2-substituted butanedioic acid ester prepared by reacting the fumaric ester in the polyaspartic acid ester solution with a cyanoacetate, a malononitrile or a 1,3-diketone in the presence of a base.

This disclosure also relates to a method for the preparation of a mixture of a polyaspartic ester and a 2-substituted butanedioic acid ester by reacting the fumaric ester in the polyaspartic acid ester solution with a cyanoacetate, or a malononitrile or a 1,3-diketone in the presence of a base in-situ. This mixture is then used to prepare polyurea coatings on reaction with polyisocyanates.

The polyaspartic ester is represented by the structure below:

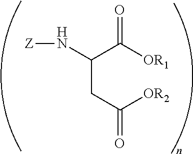

Z=a cycloalkyl or alkyl group, R₁, R₂=alkyl groups containing 1-10 carbon atoms, and n=2-4. Preferably, this polyspartic ester is obtained by a Michael reaction of an ester of maleic acid or fumaric acid with a cycloaliphatic or acyclic diamine or triamine. Preferred examples of suitable maleic and fumaric esters include diethyl maleate, dipropyl maleate, dibutyl maleate, methyl propyl maleate, ethyl propyl maleate, and the like. Preferred examples of suitable dialkyl fumarates include, diethyl fumarate, dipropyl fumarate, dibutyl fumarate, methyl propyl fumarate, ethyl propyl fumarate, and mixtures thereof.

The amine component is preferably selected from difunctional or trifunctional cycloalkyl and straight or branched chain alkyl amines. Preferred examples of suitable amines include but are not limited to 2,4′- and/or 4,4′-diaminodicyclohexylmethane, and 3,3′-dimethyl-4,4′-diaminodicyclohexylmethane. Further preferred cycloalkyl amines include Bis-(3-methyl-4-aminocyclohexyl)methane, 2,4-diamino-1-methyl cyclohexane, and 2,6-diamino-1-methyl cyclohexane, 2,4- and/or 2,6-hexahydrotolylenediamine, and 3-aminomethyl-3,5,5-trimethylcyclohexylamine. Further preferred aromatic polyamines such as 2,4- and/or 2,6-diaminotoluene, and 2,4′- and/or 4,4′-diaminodiphenyl-2,4- and/or 2,6-hexahydrotolylenediamine, are also suitable but less preferred. Further preferred straight and branched chain alkyl amines include ethylene diamine, 1,2-diaminopropane, 1,4-diaminobutane, 1,6-diaminohexane, 2,5-dimethylhexane, 2,2,4- and/or 2,4,4-trimethyl-1,6-diaminohexane, 1,11-diaminoundecane, and 1,12-diaminododecane.

Further preferred suitable trifunctional amines include 4-aminomethyl-1,8-diaminooctane (also known as triaminononane supplied by Ascend Corp.), tris-(2-aminoethyl) amine. Further preferred tetrafunctional amines, e.g., N,N,N′,N′-tetrakis-(2-aminoethyl)-1,2-ethanediamine are also suitable.

Preferably, Z is the cycloalkyl, or alkyl group attached to the amino groups of 2,4′- and/or 4,4′-diaminodicyclohexylmethane, 3,3′-dimethyl-4,4′-diaminodicyclohexylmethane, Bis-(3-methyl-4-aminocyclohexyl) methane, 2,4-diamino-1-methyl cyclohexane, 2,6-diamino-1-methyl cyclohexane, 2,4- and/or 2,6-hexahydrotolylenediamine, 3-aminomethyl-3,5,5-trimethylcyclohexylamine, 2,4- and/or 2,6-diaminotoluene, 2,4′- and/or 4,4′-diaminodiphenyl-2,4- and/or 2,6-hexahydrotolylenediamine, ethylene diamine, 1,2-diaminopropane, 1,4-diaminobutane, 1,6-diaminohexane, 2,5-dimethylhexane, 2,2,4- and/or 2,4,4-trimethyl-1,6-diaminohexane, 1,11-diaminoundecane, 1,12-diaminododecane, 4-aminomethyl-1,8-diaminooctane, or tris-(2-aminoethyl)amine.

This invention also relates to a process for preparing a polyaspartic ester/2-substituted butanedioic acid ester mixture in-situ by reacting an ester of fumaric acid or maleic acid with a polyamine and then reacting the residual ester of fumaric acid or maleic ester to completion with a cyanoacetate, or a malononitrile or a 1,3-diketone in the presence of a base. This mixture is then used to prepare polyurea coatings on reaction with polyisocyanates.

During the formation of the polyaspartic ester component, an ester of fumaric or maleic acid is reacted with the diamine or triamine in a first step of the process. In one embodiment, the molar ratio of the diamine to the maleic or fumaric ester is in the range of 1:3 to 1:2. In another embodiment, the molar ratio of the diamine to the maleic or fumaric ester is in the range of 1.0:2.5 to 1.0:2.2. In another embodiment, the molar ratio of the diamine to the maleic or fumaric ester is in the range of 1.0:2.0. In reaction of the ester of maleic or fumaric ester with a triamine, in one embodiment the molar ratio of the triamine to the maleic or fumaric ester is in the range of 1.0:4.0 to 1.0:3.0. In another embodiment, the molar ratio of the triamine to the maleic or fumaric ester is in the range of 1.0:3.5. In another embodiment, the molar ratio of the triamine to the maleic or fumaric ester is in the range of 1.0:3.0. This reaction is generally carried out in about eight hours. Preferably, the reaction takes place at a temperature of 25° C. to 100° C. The reaction may take place in the absence or in the presence of suitable solvents such as methanol, ethanol, propanol, ethyl- or butyl acetate and mixtures of these solvents. The pressure of the reaction is generally atmospheric.

In the second step of the process, the polyaspartic ester formed initially with unreacted fumarate or maleate ester is reacted with a cyanoacetate, or a malononitrile or a 1,3-diketone in the presence of a base (Michael reaction). In this process, the excess fumarate and maleate are consumed during the Michael reaction to generate a mixture of polyaspartic ester and a 2-substituted butanedioic acid ester The amount of cyanoacetate, or a malononitrile or a 1,3-diketone used is based on the amount of fumarate and maleate ester present after the initial polyaspartic ester synthesis process. This is determined by analysis of the reaction mixture by gas chromatography or similar analytical technique. Preferably, the molar ratio of fumarate and maleate ester combined to the cyanoacetate, or malononitrile or 1,3-diketone used is about 1.0. Preferably, the amount of base used in the reaction with these compounds is in the range of 0.01-1.0 molar equivalent relative to the cyanoacetate, malononitrile or 1,3-diketone. Preferably, the weight ratio of polyaspartic ester to 2-substituted butanedioic acid ester is in the range of 98:2 to 75:25. Further preferred, in some cases the ratio is 80:20 and in other cases 92:8. An example of this process is illustrated by the reaction of ethyl cyanoacetate with diethyl fumarate in the presence of the base, potassium carbonate.

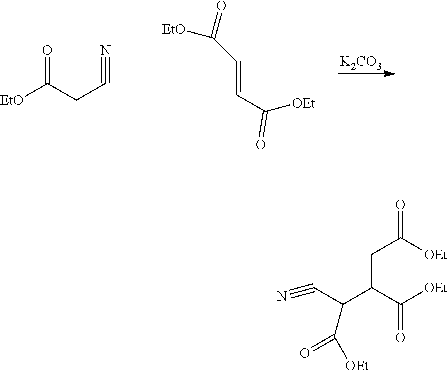 [SMILES: CCOC(=O)CC#N ; CCOC(=O)/C=C/C(=O)OCC ; "K2CO3" ; CCOC(=O)CC(C(=O)OCC)C(C#N)C(=O)OCC]

Preferably, cyanoacetates, malononitriles and 1,3-diketones that are suitable for this process are represented by the structures below:

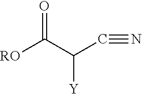

R=alkyl group of 1-12 carbon atoms or an aryl group; Y=H, alkyl group of 1-12 carbons or an aryl group.

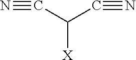

X=H, alkyl group of 1-12 carbon atoms or an aryl group.

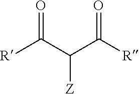

R′, R″,=alkyl group of 1-12 carbon atoms or an aryl group. Z=H, an alkyl group of 1-12 carbon atoms or an aryl group.

The base is used for deprotonation of the hydrogen atom on the carbon bearing the two electron withdrawing groups (e.g. CN, CO). Preferred bases include carbonates e.g. potassium carbonate, sodium carbonate and lithium carbonate, hydroxides e.g. sodium hydroxide, potassium hydroxide and lithium hydroxide, tertiary amines e.g. triethylamine, trimethylamine and other trialkylamines or triarylamines. Other preferred bases include cyclic amines such as tetramethyl guanidine, diazabicylcononane (DBN), diazabicycloundecane (DBU), triethylenediamine etc. In general any base that is strong enough to deprotonate the hydrogen atom of the carbon bearing the two electron withdrawing groups (CN, CO) can be used.

Preferably, the mole ratio of the base to cyanoacetate, or to malononitrile or to the 1,3-diketone is 0.01-5.0.

Preferably, the polyisocyanate component used to react with the polyaspartic ester/2-substituted butanedioic acid ester mixture include 1,4-diisocyanatobutane, 1,6-hexamethylene diisocyanate, 2,2,4- and/or 2,4,4-trimethyl-1,6-hexamethylene diisocyanate, dodecamethylene diisocyanate, 1,4-diisocyanatocyclohexane, 1-isocyanato-3,3,5-trimethyl-5-isocyanatomethylcyclohexane (IPDI), 2,4′- and/or 4,4′-diisocyanato-dicyclohexyl methane, 2,4- and/or 4,4′-diisocyanatodiphenyl methane and mixtures of these isomers with their higher homologues that are obtained in a known manner by the phosgenation of aniline/formaldehyde condensate, 2,4- and/or 2,6-diisocyanatotoluene and any mixtures of these compounds. Preferred cyclic isocyanates include diphenylmethane 4,4′-diisocyanate (MDI), diphenylmethane 2,4′-diisocyanate, 2,4- and/or 2,6-diisocyanatotoluene. Preferred aliphatic isocyanates include hexamethylene diisocyanate, isophorone diisocyanate, 2,4′- and/or 4,4′-diisocyanato-dicyclohexyl methane.

Preferred additional suitable polyisocyanate components include derivatives of the above-mentioned monomeric polyisocyanate, as is conventional in coatings technology. Preferably, these derivatives include polyisocyanate containing biuret groups as described, for example, in U.S. Pat. Nos. 3,124,605 and 3,201,372 and DE-OS 1,101,394, incorporated herein by reference in their entirety; polyisocyanate containing isocyanurate groups as described in U.S. Patent. No. 3,001,973, DE-PS 1,022,789, 1,222,067 and 1,027,394 and DE-OS 1,929,034 and 2,004,048, incorporated herein by reference in their entirety; polyisocyanate containing urethane groups as described, for instance, in DE-OS 953,012, BE-PS 752,261 and U.S. Pat. Nos. 3,394,164 and 3,644,457; polyisocyanate containing carbodiimide groups as described in DE-PS 1,092,007, U.S. Pat. No. 3,152,162 and DE-OS 2,504,400, 2,537,685 and 2,552,350, incorporated herein by reference in their entirety; and polyisocyanate containing allophanate groups as described, for example, in GB-PS 994,890, BE-PS 761,626 and NL-OS 7,102,524. Further preferred polyisocyanate also include polyisocyanate that contain uretdione groups. In one embodiment, asymmetric trimers such as those in U.S. Pat. No. 5,717,091, incorporated herein by reference in its entirety, are also suitable. Further preferred isocyanate group-containing prepolymers and semi-prepolymers based on polyisocyanate can also be used as the polyisocyanate component. In one embodiment, these prepolymers and semi-prepolymers have an isocyanate content ranging from about 0.5 to 30% by weight. In another embodiment, these prepolymers and semi-prepolymers preferably have an isocyanate content ranging from about 1 to 20% by weight. In one embodiment, these prepolymers and semi-prepolymers are prepared in a known manner by the reaction of starting materials, e.g., isocyanate-reactive compounds such as polyols, at an NCO/OH equivalent ratio of about 1.05:1 to 10:1. In another embodiment, these prepolymers and semi-prepolymers are prepared at an NCO/OH equivalent ratio of about 1.1:1 to 3:1.

The following invention is directed to the following aspects:

<1> A polyurea coating composition comprising
(A) a polyisocyanate;
(B) a polyaspartic acid ester represented by the structure below:

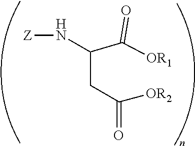

wherein Z=a cycloalkyl or alkyl group, R₁, R₂=alkyl groups containing 1-10 carbon atoms, and n=2-4; and
(C) a 2-substituted butanedioic acid ester prepared by reacting the fumaric ester in the polyaspartic acid ester solution with a cyanoacetate, a malononitrile or a 1,3-diketone in the presence of a base.
   <2> The composition of aspect <1> wherein the cyanoacetate, the malononitrile or the 1,3-diketone is selected from the group consisting of

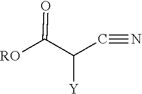

R=alkyl group of 1-12 carbon atoms or an aryl group; Y=H, alkyl group of 1-12 carbons or an aryl group;

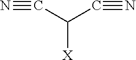

X=H, alkyl group of 1-12 carbon atoms or an aryl group; and

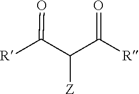

R′, R″,=alkyl group of 1-12 carbon atoms or an aryl group; Z=H, an alkyl group of 1-12 carbon atoms or an aryl group.
<3> The composition of aspect <1> wherein Z is the cycloalkyl or alkyl group attached to at least one of the amino groups of 2,4′- and/or 4,4′-diaminodicyclohexylmethane, 3,3′-dimethyl-4,4′-diaminodicyclohexylmethane, Bis-(3-methyl-4-aminocyclohexyl) methane, 2,4-diamino-1-methyl cyclohexane, 2,6-diamino-1-methyl cyclohexane, ethylene diamine, 1,2-diaminopropane, 1,4-diaminobutane, 1,6-diaminohexane, 2,5-dimethylhexane, 2,2,4- and/or 2,4,4-trimethyl-1,6-diaminohexane, 1,11-diaminoundecane, 1,12-diaminododecane, 1-amino-3,3,5-trimethyl-5-aminomethylcyclohexane, 4-aminomethyl-1,8-diaminooctane, or tris-(2-aminoethyl) amine.
<4> The composition of aspect <1> wherein the polyisocyanate is selected from the group consisting of 1,4-diisocyanatobutane, 1,6-hexamethylene diisocyanate, 2,2,4- and/or 2,4,4-trimethyl-1,6-hexamethylene diisocyanate, dodecamethylene diisocyanate, 1,4-diisocyanatocyclohexane, 1-isocyanato-3,3,5-trimethyl-5-isocyanatomethylcyclohexane, 2,4′- and/or 4,4′-diisocyanato-dicyclohexyl methane, 2,4- and/or 4,4′-diisocyanatodiphenyl methane, 2,4- and/or 2,6-diisocyanatotoluene, diphenylmethane 4,4′-diisocyanate, diphenylmethane 2,4′-diisocyanate, 2,4- and/or 2,6-diisocyanatotoluene, hexamethylene diisocyanate, isophorone diisocyanate, or 2,4′- and/or 4,4′-diisocyanato-dicyclohexyl methane.
<5> Use of a polyurea coating composition according to one of aspects <1> to <4> for the preparation of a polyurea coating.
<6> A process for preparing a polyaspartic ester/2-substituted butanedioic acid ester mixture comprising the steps of
a) reacting an ester of fumaric acid or maleic acid with a polyamine; and
b) reacting the residual ester of fumaric acid or maleic ester to completion with a cyanoacetate, a malononitrile or a 1,3-diketone.
   <7> The process of aspect <6> wherein the polyamine in step a) is a cycloaliphatic or acyclic diamine or triamine.
   <8> The process of aspect <7> wherein the cycloaliphatic or acyclic diamine or triamine is selected from the group consisting of 2,4′- and/or 4,4′-diaminodicyclohexylmethane, 3,3′-dimethyl-4,4′-diaminodicyclohexylmethane, Bis-(3-methyl-4-aminocyclohexyl) methane, 2,4-diamino-1-methyl cyclohexane, 2,6-diamino-1-methyl cyclohexane, 2,4- and/or 2,6-hexahydrotolylenediamine, 3-aminomethyl-3,5,5-trimethylcyclohexylamine, ethylene diamine, 1,2-diaminopropane, 1,4-diaminobutane, 1,6-diaminohexane, 2,5-dimethylhexane, 2,2,4- and/or 2,4,4-trimethyl-1,6-diaminohexane, 1,11-diaminoundecane, and 1,12-diaminododecane.
   <9> The process of aspect <6> wherein the cyanoacetate, the malononitrile or the 1,3-diketone is selected from the group consisting of

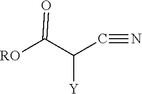

R=alkyl group of 1-12 carbon atoms or an aryl group; Y=H, alkyl group of 1-12 carbons or an aryl group;

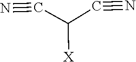

X=H, alkyl group of 1-12 carbon atoms or an aryl group; and

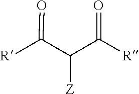

R′, R″,=alkyl group of 1-12 carbon atoms or an aryl group; Z=H, an alkyl group of 1-12 carbon atoms or an aryl group.
<10> The process of aspect <7> wherein the molar ratio of diamine to the ester of fumaric acid or maleic acid is in the range of 1:3 to 1:2.
<11> The process of aspect <7> wherein the molar ratio of triamine to the ester of fumaric acid or maleic acid is in the range of 1:4 to 1:3.
<12> The process of aspect <6> wherein the molar ratio of the residual ester of fumaric acid or maleic acid to the cyanoacetate, the malononitrile or the 1,3-diketone is about 1.0.
<13> The process of aspect <6> wherein the weight ratio of polyaspartic ester to 2-substituted butanedioic acid ester is in the range of 98:2 to 75:25.

### EXAMPLES

Example 1. Preparation of polyaspartic ester mixture from diethyl maleate, 4,4′-diaminodicyclohexylmethane (PACM) and Michael reaction of residual diethyl fumarate with ethyl cyanoacetate/K2CO3 (molar eq. of ethyl cyanoacetate and K₂CO₃).

A 2L glass reactor equipped with a N₂ inlet tube, thermocouple and addition funnel was charged with 4,4′-diaminodicyclohexylmethane (PACM) (470.72g, 2.0 mole) and heated to 80° C. Diethyl maleate (688.8 g, 4 mole) was slowly added while maintaining the temperature at 80-85° C. A mixture of polyaspartic ester and diethyl fumarate was obtained.

An aged sample (100 g) with a diethyl fumarate concentration of 2.31 wt % (0.013 mole) as indicated by gas chromatography was treated with ethyl cyanoacetate (0.013 mole, 1.47 g) and potassium carbonate (0.013 mole, 1.80 g) at 85° C. for 3 h under a N₂ atmosphere then brought to ambient temperature. The diethyl fumarate concentration was reduced to 0.26 wt. %.
Example 2. Preparation of polyaspartic ester mixture from diethyl maleate, 4,4′-diaminodicyclohexylmethane (PACM) and Michael reaction of residual diethyl fumarate with ethyl cyanoacetate/tetramethylguanidine (catalytic amount).

An aged sample (100 g) prepared as above with a diethyl fumarate concentration of 2.31 wt % (0.013 mole) as indicated by gas chromatography was treated with ethyl cyanoacetate (0.013 mole, 1.47 g) and tetramethylguanidine (0.0013 mole, 0.15 g) at 40°° C. for 2h and ambient temperature for 7 d under a N₂ atmosphere. The diethyl fumarate concentration was undetected

Example 3. Preparation of polyaspartic ester mixture from diethyl maleate, 4,4′-diaminodicyclohexylmethane (PACM) and Michael reaction of residual diethyl fumarate with ethyl cyanoacetate/DBU (catalytic amount).

An aged sample (100 g) prepared as above with a diethyl fumarate concentration of 2.31 wt % (0.013 mole) as indicated by gas chromatography was treated with ethyl cyanoacetate (0.013 mole, 1.47 g) and DBU (0.0013 mole, 0.20 g) at ambient temperature for 7 d under a N₂ atmosphere. The diethyl fumarate concentration was undetected.

Example 4. Preparation of polyaspartic ester mixture from diethyl maleate, 4,4′-diaminodicyclohexylmethane (PACM) and Michael reaction of residual diethyl fumarate with malononitrile/K₂CO₃.

An aged sample (100 g) prepared as above with a diethyl fumarate concentration of 2.31 wt % (0.013 mole) as indicated by gas chromatography was treated with malononitrile (0.013 mole, g) and K₂CO₃ (0.0013 mole, 1.80 g) at 85° C. then cooled to ambient temperature. The diethyl fumarate concentration was 0.47 wt %.

Example 5. Preparation of polyaspartic ester mixture from diethyl maleate, 4,4′-diaminodicyclohexylmethane (PACM) and Michael reaction of residual diethyl fumarate with acetylacetone/K₂CO₃.

An aged sample (100 g) prepared as above with a diethyl fumarate concentration of 2.31 wt % (0.013 mole) as indicated by gas chromatography was treated with acetylacetone (0.013 mole, g) and K₂CO₃ (0.0013 mole, 1.80 g) at 85° C. then cooled to ambient temperature. The diethyl fumarate concentration was 1.18 wt %.

Example 6. Preparation of polyaspartic ester mixture from diethyl maleate, 4,4′-diaminodicyclohexylmethane (PACM) and Michael reaction of residual diethyl fumarate with ethyl acetoacetate/K2CO3.

An aged sample (100g) prepared as above with a diethyl fumarate concentration of 2.31 wt % (0.013 mole) as indicated by gas chromatography was treated with ethyl acetoacetate (0.013 mole, g) and K₂CO₃ (0.0013 mole, 1.80 g) at 85° C. then cooled to ambient temperature. The diethyl fumarate concentration was 1.82 wt %.

### Example 7.

Coating mixtures were prepared by mixing the product from Examples 1 to 6 separately with a polyisocyanate, hexamethylene diisocyanate trimer at a stoichiometric ratio of 1.05 NCO to amine. 138 grams of the amine was added to the container followed by 100 grams of the hexamethylene diisoycanate trimer, Vestanat HT2500/100 (21.8% NCO). The materials in the container were hand mixed for about 1 to 2 minutes with a spatula to form a homogeneous mixture. The mixtures were then used for different testing including viscosity measurements, dry times and Shore D hardness. A control mixture of polyaspartic ester/aspartic ester mixture from diethyl maleate, 4,4′-diaminodicyclohexylmethane (PACM) with the same polyisocyanate was also prepared for side to side comparison.

### Example 8.

Viscosity was measured on the neat amine composition prepared in Examples1 to 6 alongside the control using ASTM D2196-10 test method A. The mix viscosity and cure profile of the samples prepared in Example 7 were also measured. The cure profile recorded is the amount of time it takes for the viscosity of the sample to reach 12,000 cP. All the viscosity data are shown in Table 1a and 1b. Viscosity testing was conducted on a Brookfield Viscometer, model RV-DVIII with a thermoset accessory and a small sample adapter. The chamber required 12 ml of sample and 27 spindle was used for measurements. All viscosity testings were conducted at 25° C.

### Example 9.

The dry times of the mixture prepared in Example 7 were also measured. A thin coating of about 150 micron was applied by a draw down bar on a 12″ by 1″ glass slide and placed on BK drying time recorder. The thin needle recording time was set at 2 hours. A set to touch (stage 1) time and tack free (stage 2) time was determined on a dried film by using ASTM D5895. The dry time data are shown in Table 1a and 1b. All dry time measurements were conducted at 22° C./50% RH.

### Example 10.

Shore D hardness were measured on mixture composition prepared in Example 7. A wet mixture was poured into a small circular mold to form a ⅛″ coating once it hardened. The hardness was measured on the coating using a Shore D durometer from PTC instruments (model 307L) at 4, 6 and 24 hours using ASTM D2240 method. The shore D hardness data are shown in Table 1. All shore D measurements were conducted at 22C/50% RH.

TABLE 1a
Coating mixture properties of Polyaspartic
amines reacted with polyisocyanate.

| Polyisocyanate | Vestanat | Vestanat | Vestanat |
|---|---|---|---|
| (HDI Trimer) | HT2500/100 | HT2500/100 | HT2500/100 |
| % NCO | 21.8% | 21.8% | 21.8% |

| | | | |
|---|---|---|---|
| | Polyaspartic | Polyaspartic | Polyaspartic |
| Amine Curing agent | (Control) | (Example 1) | (Example 2) |

Brookfield Viscometer (ASTM D2196-10) - Spindle
27; Torque 10-90%; 25° C.

| | | |
|---|---|---|
| Neat Viscosity (cP): | | |
| Mix Viscosity (cP): | 2967 | 2700 |
| Time to 12,000 cP (min): | 15.5 | 26.0 |

Dry time (ASTM D5895) - (2 hr. Setting) - 22° C.
and 50% Relative humidity

| | | |
|---|---|---|
| Set to touch- | 6 | 6 |
| Stage 1 (min): | | |
| Tack free- | 13 | 12 |
| Stage 2 (min): | | |

Shore D Hardness (ASTM D2240) - 22° C.
and 50% Relative humidity

| | | | |
|---|---|---|---|
| 4 | hrs. | 60 | 55 |
| 6 | hrs. | 63 | 63 |
| 24 | hrs. | 66 | 66 |

TABLE 1b
Coating mixture properties of Polyaspartic amines reacted with polyisocyanate.

| Polyisocyanate | Vestanat | Vestanat | Vestanat | Vestanat |
|---|---|---|---|---|
| (HDI Trimer) | HT2500/100 | HT2500/100 | HT2500/100 | HT2500/100 |
| % NCO | 21.8% | 21.8% | 21.8% | 21.8% |

| | | | | |
|---|---|---|---|---|
| | Polyaspartic | Polyaspartic | Polyaspartic | Polyaspartic |
| Amine Curing agent | (Example3) | (Example 4) | (Example 5) | (Example 6) |

Brookfield Viscometer (ASTM D2196-10) - Spindle 27; Torque 10-90%; 25° C.

| | | | |
|---|---|---|---|
| Neat Viscosity (cP): | | | |
| Mix Viscosity (cP): | 4433 | 2867 | 2717 |
| Time to 12,000 cP (min): | 12 | 21.5 | 21 |

Dry time (ASTM D5895) - (2 hr. Setting) - 22° C. and 50% Relative humidity

| | | | |
|---|---|---|---|
| Set to touch- | 6 | 6 | 6 |
| Stage 1 (min): | | | |
| Tack free- | 11 | 12 | 13 |
| Stage 2 (min): | | | |

Shore D Hardness (ASTM D2240) - 22° C. and 50% Relative humidity

| | | | | |
|---|---|---|---|---|
| 4 | hrs. | 48 | 49 | 52 |
| 6 | hrs. | 60 | 60 | 63 |
| 24 | hrs. | 65 | 64 | 63 |

1. A polyurea coating composition comprising
(A) a polyisocyanate;
(B) a polyaspartic acid ester represented by the structure below: 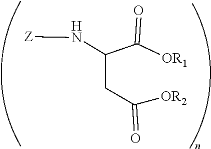
wherein Z=a cycloalkyl or alkyl group, R₁, R₂=alkyl groups containing 1-10 carbon atoms, and n=2-4; and
(C) a 2-substituted butanedioic acid ester prepared by reacting the fumaric ester in the polyaspartic acid ester solution with a cyanoacetate, a malononitrile or a 1,3-diketone in the presence of a base.

2. The composition of claim 1 wherein the cyanoacetate, the malononitrile or the 1,3-diketone is selected from the group consisting of 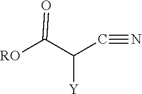
R=alkyl group of 1-12 carbon atoms or an aryl group; Y=H, alkyl group of 1-12 carbons or an aryl group; 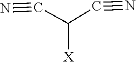
X=H, alkyl group of 1-12 carbon atoms or an aryl group; and 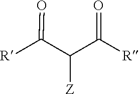
R′, R″,=alkyl group of 1-12 carbon atoms or an aryl group; Z=H, an alkyl group of 1-12 carbon atoms or an aryl group.

3. The composition of claim 1 wherein Z is the cycloalkyl or alkyl group attached to at least one of the amino groups of 2,4′- and/or 4,4′-diaminodicyclohexylmethane, 3,3′-dimethyl-4,4′-diaminodicyclohexylmethane, Bis-(3-methyl-4-aminocyclohexyl) methane, 2,4-diamino-1-methyl cyclohexane, 2,6-diamino-1-methyl cyclohexane, ethylene diamine, 1,2-diaminopropane, 1,4-diaminobutane, 1,6-diaminohexane, 2,5-dimethylhexane, 2,2,4- and/or 2,4,4-trimethyl-1,6-diaminohexane, 1,11-diaminoundecane, 1, 12-diaminododecane, 1-amino-3,3,5-trimethyl-5-aminomethylcyclohexane, 4-aminomethyl-1,8-diaminooctane, or tris-(2-aminoethyl)amine.

4. The composition of claim 1 wherein the polyisocyanate is selected from the group consisting of 1,4-diisocyanatobutane, 1,6-hexamethylene diisocyanate, 2,2,4- and/or 2,4,4-trimethyl-1,6-hexamethylene diisocyanate, dodecamethylene diisocyanate, 1,4-diisocyanatocyclohexane, 1-isocyanato-3,3,5-trimethyl-5-isocyanatomethylcyclohexane, 2,4′- and/or 4,4′-diisocyanato-dicyclohexyl methane, 2,4- and/or 4,4′-diisocyanatodiphenyl methane, 2,4- and/or 2,6-diisocyanatotoluene, diphenylmethane 4,4′-diisocyanate, diphenylmethane 2,4′-diisocyanate, 2,4- and/or 2,6-diisocyanatotoluene, hexamethylene diisocyanate, isophorone diisocyanate, or 2,4′- and/or 4,4′-diisocyanato-dicyclohexyl methane.

5. A method of preparing a polyurea coating comprising reacting a polyaspartic acid ester represented by the structure below: 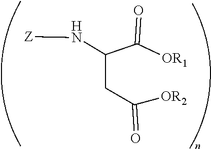
wherein Z=a cycloalkyl or alkyl group, R₁, R₂=alkyl groups containing 1-10 carbon atoms, and n=2-4,
with a 2-substituted butanedioic acid ester prepared by reacting the fumaric ester in the polyaspartic acid ester solution with a cyanoacetate, a malononitrile or a 1,3-diketone in the presence of a base to form a mixture, and reacting the mixture with a polyisocyanate.

6. A process for preparing a polyaspartic ester/2-substituted butanedioic acid ester mixture comprising the steps of
a) reacting an ester of fumaric acid or maleic acid with a polyamine; and
b) reacting the residual ester of fumaric acid or maleic ester to completion with a cyanoacetate, a malononitrile or a 1,3-diketone.

7. The process of claim 6 wherein the polyamine in step a) is a cycloaliphatic or acyclic diamine or triamine.

8. The process of claim 7 wherein the cycloaliphatic or acyclic diamine or triamine is selected from the group consisting of 2,4′- and/or 4,4′-diaminodicyclohexylmethane, 3,3′-dimethyl-4,4′-diaminodicyclohexylmethane, Bis-(3-methyl-4-aminocyclohexyl) methane, 2,4-diamino-1-methyl cyclohexane, 2,6-diamino-1-methyl cyclohexane, 2,4- and/or 2,6-hexahydrotolylenediamine, 3-aminomethyl-3,5,5-trimethylcyclohexylamine, ethylene diamine, 1,2-diaminopropane, 1,4-diaminobutane, 1,6-diaminohexane, 2,5-dimethylhexane, 2,2,4- and/or 2,4,4-trimethyl-1,6-diaminohexane, 1,11-diaminoundecane, and 1,12-diaminododecane.

9. The process of claim 6 wherein the cyanoacetate, the malononitrile or the 1,3-diketone is selected from the group consisting of 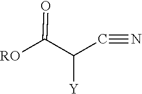
R=alkyl group of 1-12 carbon atoms or an aryl group; Y=H, alkyl group of 1-12 carbons or an aryl group; 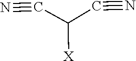
X=H, alkyl group of 1-12 carbon atoms or an aryl group; and 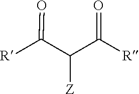
R′, R″,=alkyl group of 1-12 carbon atoms or an aryl group; Z=H, an alkyl group of 1-12 carbon atoms or an aryl group.

10. The process of claim 7 wherein the molar ratio of diamine to the ester of fumaric acid or maleic acid is in the range of 1:3 to 1:2.

11. The process of claim 7 wherein the molar ratio of triamine to the ester of fumaric acid or maleic acid is in the range of 1:4 to 1:3.

12. The process of claim 6 wherein the molar ratio of the residual ester of fumaric acid or maleic acid to the cyanoacetate, the malononitrile or the 1,3-diketone is about 1.0.

13. The process of claim 6 wherein the weight ratio of polyaspartic ester to 2-substituted butanedioic acid ester is in the range of 98:2 to 75:25.
